(19) United States
(12) Patent Application Publication
(10) Pub. No.: US 2008/0281219 A1
(43) Pub. Date: Nov. 13, 2008

(54) Method and System for Assessing Lung Condition and Managing Mechanical Respiratory Ventilation

(75) Inventors: Yael Glickman, Haifa (IL); Igal Kushnir, Pardes Hana (IL); Smith Jean, Philadelphia, PA (US); Phillip Dellinger, Philadelphia, PA (US)

Correspondence Address: OLIFF & BERRIDGE, PLC, P.O. BOX 320850, ALEXANDRIA, VA, 22320-4850, US

(73) Assignee: DeepBreeze Ltd., Industrial Park Or Akiva (IL)

(21) Appl. No.: 12/035,171

(22) Filed: Feb. 21, 2008

Related U.S. Application Data
(60) Provisional application No. 60/907,605, filed on Apr. 11, 2007.

(51) Int. Cl. A61B 5/08

(52) U.S. Cl. 600/533; 600/529

(57) ABSTRACT

The present invention discloses a novel non-invasive, bedside system and method to monitor parameters associated lung changes. A novel approach for monitoring the operation of the respiratory system of a subject is provided. There is also provided a method for objectively evaluating the benefit of one mode of ventilation over another, and for assessing the differences in regional lung vibration during different modes of mechanical ventilation. The method comprises recording one or more signals from the subject, the signal varying in time according to operation of the respiratory system; and; processing the recorded signals to obtain a predetermined functional thereof presenting one or more time-varying energy functions of the subject, an abnormality in the one or more energy functions being indicative of a suspected abnormality in the operation of the respiratory system. The signals may be acoustic signals recorded by a plurality of acoustic sensors placed over the subject's thorax or back, and the at least one time-varying energy function is obtained from one or more specific regions of lung or by summing/averaging the time-dependent acoustic signals of the plurality of sensors indicative of the whole lungs.

### FIELD OF THE INVENTION

The present invention relates to method and system for use in assessing lung condition and managing of mechanical respiratory ventilation.

### REFERENCES

The following references are considered to be pertinent for the purpose of understanding the background of the present invention:

[1] Baker A B, Colliss J E, Cowie R W: Effect of varying inspiratory flow waveform and time in intermittent positive pressure ventilation. Various physiological variables. Br J Anaesth 1977, 49:1221-1234.

Campbell R S, Davis B R: Pressure-controlled versus volume controlled ventilation: does it matter? Respir Care 2002, 47:416-424.

Chiumello D, Pelosi P, Calvi E: Different modes of assisted ventilation in patients with acute respiratory failure. Eur Respir J 2002, 20:925-933.

Kallet R H, Alonos J A, Morabito D J: The effects of PC vs VC assisted ventilation in acute lung injury and ARDS. Respir Care 2000, 45:1085-1096.

Mead J, Takishina T, Leith D: Stress distribution in lungs: a model of pulmonary elasticity. J Appl Physiol 1970, 28:596-608.

Rappaport S H, Shipner R, Yoshihara G: Randomized, prospective trial of pressure-limited versus volume-controlled ventilation in severe respiratory failure. Crit Care Med 1994, 22:22-32.

Davis K, Branson R, Campbell R, Porembka D: Comparison of volume control and pressure control ventilation: is flow waveform the difference? J Trauma 1996, 41:808-814.

### BACKGROUND OF THE INVENTION

Respiratory problems ail infants and adults alike. Some common lung diseases or conditions include asthma, Chronic Obstructive Pulmonary Disease (COPD), regional collapse (atelectasis), consolidation (e.g. pneumonia), interstitial edema, focal lung disease (e.g. tumour) and global lung disease (e.g. emphysema). In severe cases, respiratory abnormality is usually treated with respiratory ventilation, e.g. mechanical ventilation. Generally, respiratory ventilation (invasive or non-invasive) is a method to mechanically assist or replace spontaneous breathing when patients cannot do so on their own, and may in some cases be done so after invasive intubation with an endotracheal or tracheostomy tube through which air is directly delivered. Lung injury associated with mechanical ventilation causes many infants to develop chronic lung disease, which is characterized by persisting inflammatory and fibrotic changes. Adults are also afflicted with ventilator-induced respiratory sequelae and injury such as pneumonia or barotrauma.

Respiratory ventilators are used in healthcare to provide mechanical ventilation to subjects in order to assist, or in some cases replace spontaneous breathing. Mechanical ventilation is often critical for saving life in intensive care medicine as well as during anesthesia.

Respiratory ventilators operate in a variety of operational modes including volume control (VC), assist pressure control (PC) and pressure support (PS) modes.

There is no conclusive evidence that one mode of ventilation is better than another. With most ventilators, selection of VC requires setting of tidal volume (V_{T}), respiratory rate (RR), and inspiratory flow rate or time. In PC mode, pressure, RR, and inspiratory time are set. In PS mode, the level of inspired pressure is set and all other parameters are determined by the patient.

The major differences between VC and the other two modes are the inspiratory flow and pressure waveforms [1-3]. In VC mode, the pressure rises throughout inspiration and the inspiratory flow can be constant, decelerating, or sine-patterned. On the other hand, both PC and PS have a square pressure waveform and a decelerating inspiratory flow pattern, in which the inspiratory flow rate is high at the beginning and decreases with time. Although some studies have shown differences in work of breathing [4], lung mechanics [5, 6], and gas exchange [6, 7] in patients ventilated with these different waveforms, no consistent reproducible findings have demonstrated the benefit of one mode of ventilation over another. In fact, modes are routinely chosen by the personal preference of the treating physician or respiratory therapist.

Acoustic-based system for monitoring respiratory function are disclosed in U.S. Pat. No. 6,887,208, WO 05/74799 and WO 06/043278, all assigned to the assignee of the present patent application.

### GENERAL DESCRIPTION

There is a need in the art in objectively evaluating the benefit of one mode of ventilation over another, and in assessing the differences in regional lung vibration during different modes of mechanical ventilation. Moreover, there is a need to provide a non-invasive, bedside system and method to monitor parameters associated lung changes.

It should be noted that, in addition to the mode, other mechanical ventilation parameters such as positive end-expiratory pressure (PEEP), respiratory rate, inspiratory pressure, pressure support, tidal volume, etc. might need to be adjusted. It should also be noted that application of different levels of PEEP may have a significant impact on ventilator-induced lung injury. Higher PEEP is associated with a greater risk of barotrauma in mechanically ventilated patients with acute lung injury or acute respiratory distress syndrome (ALI/ARDS).

In accordance with the present invention a novel approach for monitoring the operation of the respiratory system of a subject is provided. The method comprises recording one or more signals from the subject, the signal varying in time according to operation of the respiratory system; and; processing the recorded signals to obtain a predetermined functional thereof presenting one or more time-varying energy functions of the subject, an abnormality in the one or more energy functions being indicative of a suspected abnormality in the operation of the respiratory system.

The signals may be acoustic signals recorded by a plurality of acoustic sensors placed over the subject's thorax or back. The at least one time-varying energy function may be obtained from one or more specific regions of lung; and the method may utilize summing or averaging the time-dependent acoustic signals of the plurality of sensors indicative of the whole lungs.

The time-varying energy function(s) may be displayed numerically, in the form of a graph, and/or in the form of a still or dynamic digital image (succession of still images or frames). It should be noted that the recordings may be saved as both dynamic images and still images, which can be analyzed either as a whole or according to specific regions (left, right, upper, middle, and lower lung). The still or dynamic digital image is indicative of the regional distribution of vibrations in the lungs. The dynamic image enables the analyzing of the intensity and distribution of vibration within lungs in real-time.

In some embodiments, at least one energy function graph is used for appropriate selection of a still image in a dynamic image recording.

The energy function may be used for monitoring activity of the respiratory system, detecting or diagnosing pathologies of the respiratory system, and others. Monitoring the energy function, e.g. displayed on a monitoring screen, plotted on paper or displayed in any other manner, may be useful as a tool for diagnosing abnormalities/changes of the patient condition (e.g. respiratory system). It should be noted that lung sounds are generally generated by turbulent air and vibrations within the airways. Lung vibrations are produced primarily by airflow, and disease may modify vibrations detected on the chest wall. This turbulence is increased as airflow in the large- and medium-size airways reaches a critical velocity. The vibrations are affected by the structural and functional properties of the lungs and can exhibit responses that may vary in frequency, intensity, space and time. The resulting sound energy is transmitted to the skin, after filtering by the lungs and chest wall. Pathologic processes such as lung infiltrates are expected to decrease the transmission of these sounds. Therefore, the present invention may provide an assessment of lung disease in patients.

The term “energy function” used herein refers to some type of processing of the measured signals (e.g., acoustic or electrical signals) being indicative of ‘energy’ or the amplitude of the signal associated with respiration (e.g. acoustic or vibration energy), or some type of transformation between the recorded (measured) signals and the associated ‘energy’ (this should be neither confused with nor limited to the mathematical meaning of the term ‘energy’). The acoustic or vibration energy is generated in the lungs and transmitted to the surface of the chest during respiration and/or mechanical ventilation.

The invention is applicable to a wide variety of signals which may be recorded from a subject which are indicative of the function of the respiratory system. In accordance with one embodiment of the invention, the signals are acoustic signals recorded, which may be by the use of a plurality of acoustic sensors placed over the subject's thorax, for example, employing the method or system described in U.S. Pat. No. 6,887,208 and International publication WO 05/74799, the contents of which are incorporated herein by reference. However, the invention is not limited to such a method and system and a variety of other methods for recording acoustic signals indicative of the function of the respiratory system are also possible as a basis for generating the energy function in accordance with the invention. Furthermore, the invention is not limited to acoustic signals and a variety of other signals, including such obtained from bio impedance measurements and others may be applicable as a basis for generating said energy function.

In some embodiments of the invention, the measured signals (e.g. acoustic or electrical signals) being indicative of ‘energy’ or the amplitude of the signal associated with respiration (e.g. acoustic or vibration energy), are processed in the form of one or more time-varying respiration-related signals from a subject.

The term “monitoring” used herein signifies collecting and processing signals from a subject and generating the energy function; and possibly also further analysis of the energy function and generating corresponding data, which may be used for example for operating a therapeutic treatment tool. The latter may be a respiratory ventilator, e.g. mechanical ventilator.

In some embodiments, the dynamic image is created from a series of gray-scale still images or frames (each of which may represent 0.17 seconds of vibration energy recording). The result is a movie depicting a sense of air movement in the lungs. The method of the present invention may comprise displaying a ventilator waveform. The ventilator waveform is selected from pressure, flow and volume waveforms. The method may comprise synchronizing the ventilator waveform and the energy function and displaying the ventilator waveform together with the energy function.

When imaging a mechanically ventilated patient, a flow sensor is placed in the tubing between the patient and the ventilator, allowing flow and pressure waveforms to be synchronized with the image and displayed. The image also displays the percentage contribution of lung regions (left, right and upper, middle, lower) to the total vibration signal.

The results of the processing can be used for controlling various medical procedures affecting the patient's respiratory system, such as mechanical respiratory ventilation, inhalation, physiotherapy, etc. The inventors have found that the energy function provides an objective method to assess the effectiveness of therapeutic intervention, even on critically ill patients with acute respiratory difficulties. Moreover, the energy function provides an objective method to assess the changes in mechanical ventilation settings by comparing image and quantification data such as the weighted pixel count of image. The results of the processing of the energy function may quantify the lung vibration in a particular region of interest by using a quantification method such as determining the percentage contribution of lung regions or the weighted pixel count of image.

Digital analyses of images reveals that the percentage of weighted pixel counts and the percentage of the total vibration are reduce or increased in patient having affected lungs. Normalization may be applied to a predetermined range of frames. Within a frame, the areas with the highest vibration energy are represented as black in a gray-level scale and the areas with the lowest vibration energy are represented as light gray. For example, areas of a frame are white if their energy is below a signal-to-noise threshold determined by the control unit. The data presentation unit displays a video containing those normalized frames in shades of gray which reflect the intensity of vibration at each stage of the respiratory cycle.

In some embodiments of the invention, the monitoring of the respiratory system of a subject may be used in feedback mode in which it makes use of such a energy function in order to optimize the management of spontaneously breathing patients with lung pathologies as well as mechanically ventilated subjects under forced or assisted ventilation treatment, for example in the operating room, in intensive care units, etc. It was found in accordance with the invention that this energy function provides an easily identifiable measure in order to select the optimized ventilation mode, PEEP level, pressure level, etc. Therefore, the control of the mechanical ventilator may comprise changing between different ventilator settings such as ventilation modes, respiratory rate, inspiratory pressure, pressure support level, tidal volume, flow rates, rise times, I:E ratios, pressure limits, inspiratory times, and levels of PEEP.

In some embodiments, the processing of the one or more energy functions comprises determining a degree of correlation between one or more parameters of the energy function and one or more corresponding parameters of certain reference energy function. In the management of ventilation in accordance with the invention, the ventilation may be controlled so as to achieve certain correlation between the energy function of a ventilated subject to a reference function such that of self breathing subjects (e.g. correlation of geographical distribution and/or intensity of energy, synchronization and/or balance between lungs, signal periodicity, signal symmetry, etc.). Such correlation may be with the energy function of the same subject under non-ventilated conditions or to that of healthy individuals.

The results of said processing of the energy function may be used for optimizing the operational mode of mechanical ventilation or selecting optimized parameters set for a specific mode. The one or more optimized parameters set for a specific mode include at least one of the following: respiratory rate, inspiratory pressure, pressure support level, tidal volume, levels of PEEP.

The ventilation parameters can also be optimized by comparing different measurements at different settings of mechanical ventilation in the same patient under different conditions (different modes, different levels of PEEP, etc).

In some embodiments, the different modes of ventilation are objectively evaluated by different geographical distribution of vibration in the lung (i.e. different fill of ventilated lung within the lung region). The regional distribution of vibration energy is calculated for the frames of interest. The percentage changes in vibration energy within the lower lung region (two lower rows of sensors), the middle lung region (two middle rows), and the upper lung region (two upper rows) are calculated and then compared among different modes of mechanical ventilation. The measurement providing best geographical distribution of energy, best synchronization and/or energy balance between the lungs, best signal periodicity and/or symmetry may represent the optimal measurement for the patient. According to some embodiments, the energy function is measured on patients on assist volume control, assist pressure control, and pressure support modes of mechanical ventilation with constant tidal volumes (V_{T}). Images and vibration intensities of various lung regions at maximal inspiration can be analyzed. The vibration generated by airflow in a lung ventilated with different modes of mechanical ventilation (MV): VC, PC, and PS can be compared.

As indicated above, the data may be displayed as graph, as non-dynamic image(s) (still images) or as dynamic images indicative of the distribution of vibration within the lung during the respiratory process. When the energy function is displayed in form of a graph, particularly such pertained from recorded sound signals, the energy function recorded from an healthy subject, has two distinct peaks, one representing the inspiration and the other—the expiration of the lungs. These peaks are distinct and normally appear one after the other in a periodical manner. In the case of an abnormality, the distinct appearance of two peaks in the energy function may be disrupted, as well as their periodical appearance and/or the length of inspiration/expiration events and/or a ratio between them, all of which may serve as a sign of an abnormality in the respiratory system.

It should be noted that still images at maximum inspiration energy are one of the most suitable form for displaying the data in a non-dynamic form; however, the dynamic image also provides additional information on distribution of vibration energy throughout the respiratory cycle. Different processing methods may be used to assess the regional distribution of vibration in the lungs: image analysis and raw numerical data calculation. The image analysis may comprise characterizing different modes of ventilation or different parameter sets for a specific mode of ventilation by different geographical distribution of vibration in the lung.

The processing of the energy function may also comprises extracting a maximal energy frame (MEF) indicative of a frame providing the most information on the distribution of lung vibrations in a selected range of frames. The processing may comprise several stages of filtering to select a specific frequency band. The filtered output signal frequencies may be presented as a gray-scale coded dynamic image, consisting of a series of frames (e.g. 0.17 second frames), and as a table featuring the percentage contribution of each lung to the total vibration signal. The dynamic imaging technique displays energy of lung sounds generated during the respiratory cycle as a real-time structural and functional image of the respiration process. This novel technique of imaging and featuring distribution of vibration enables to study the intensity and distribution of vibration within the lungs in real time. This technique is non-invasive and displays airflow-induced vibrations as well as total and regional graphs of vibration energy. The dynamic image obtained in an individual patient provides information on whether a particular distribution of vibration signified better overall ventilation or oxygenation in that patient.

There is also provided a system for monitoring the respiratory system of a subject. The monitoring system comprises a control unit for receiving data indicative of one or more respiration-related signals, and configured and operable for processing the received data and generating at least one corresponding time-varying energy function and displaying said energy function, and being configured and operable for using said at least one corresponding time varying energy function for determining at least one of the following: a condition of the respiratory system, an optimal operational mode or optimal parameters set for a specific operational mode of a ventilation system being applied to a subject.

The control unit is configured to be connectable (via wires or wireless signal transmission) to an appropriate sensing unit (e.g. acoustic sensors arrangement). The sensing unit comprises one or more sensors for recording corresponding one or more respiration-related signals from the subject and generating data indicative thereof.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a monitoring system, in accordance with one embodiment of the invention;

FIG. 2 shows a representative energy function graph of a healthy spontaneously breathing individual (I: inspiration phase and E: expiration phase);

FIGS. 3A and 3B exemplify the energy function graph recordings before and after an inhalation treatment, respectively, from an asthmatic patient;

FIGS. 3C and 3D exemplify the energy function graph recordings before and after a physiotherapy treatment, respectively, from a spontaneously breathing individual;

FIGS. 4A to 4D show the measured energy function graphs for the same patient under four different modes of mechanical ventilation, squared volume control, decelerating volume control, pressure control and pressure support modes, respectively;

FIGS. 5A to 5C exemplify the energy function graphs of a patient ventilated with three different modes of mechanical ventilation, squared volume control, pressure control and pressure support modes, respectively;

FIG. 6 shows the pressure, air flow and energy function graphs for another patient under squared volume control ventilation mode with hold;

FIGS. 7A and 7B exemplify two energy function graphs recordings obtained from the same patient while under squared volume control and pressure support ventilation modes, respectively;

FIGS. 8 and 9 show two examples of the pressure, air flow, and the energy function graph for the cases of squared volume control and pressure control ventilation modes, respectively;

FIG. 10A exemplifies a vibration response image and FIG. 10B exemplifies a graph represented the average vibration energy as a function of time extracted from the same measured data than the vibration response image of FIG. 10A;

FIGS. 11A-11D illustrate examples of frame selection in various vibration response imaging waveform patterns. The dot on the waveform represents the area from which the maximal energy frame is chosen for analysis;

FIGS. 12A-12C illustrate still images at peak inspiration on various modes of mechanical ventilation and FIG. 12D illustrates the quantification of the image in a particular region of interest (lower lungs);

FIGS. 13A-13E illustrate the effects on PEEP changes on the still image and graph at peak inspiration.

### DETAILED DESCRIPTION OF EXEMPLARY EMBODIMENTS

The specific exemplary embodiment described below makes use of an energy function displayed in the form of a time graph and/or dynamic digital image, obtained through recording of acoustic signals, preferably in a manner as described in U.S. Pat. No. 6,887,208 and in International publication WO 05/74799. As will be appreciated, this is an exemplary embodiment and the invention is not limited thereto.

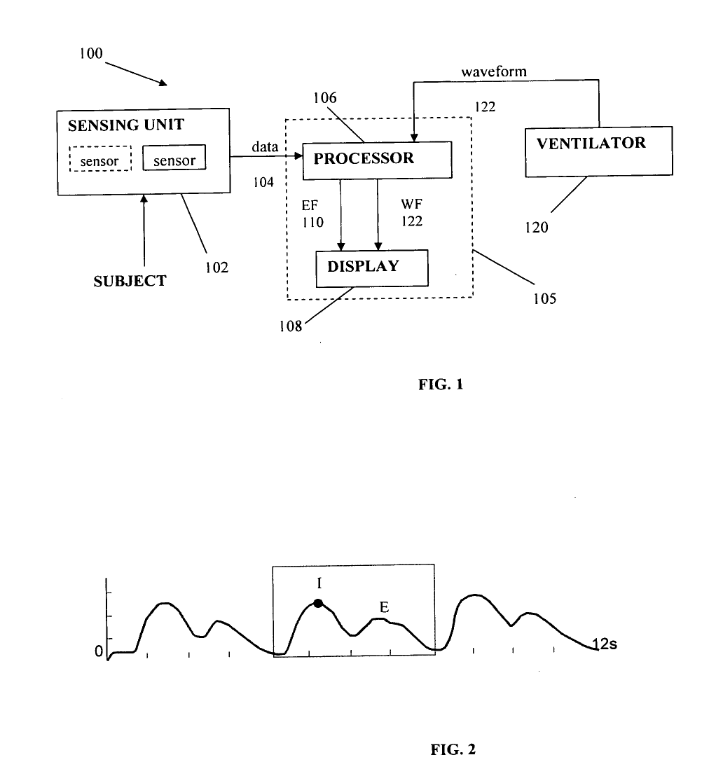

FIG. 1 shows a monitoring system 100 for analyzing signals of the respiratory system of a subject in accordance with an embodiment of the invention. The monitoring system 100 is aimed at controlling the operation of a certain therapeutic procedure, which in this specific but not limiting example of FIG. 1 is mechanical respirator ventilation. Accordingly, the system 100 is associated with a mechanical ventilator 120 (constituting a therapeutic treatment tool). The system 100 is associated with a sensing unit 102 including one or more sensors, which may, for example, be acoustic sensor(s), as described in U.S. Pat. No. 6,887,208 and WO 05/74799. The sensor(s) is/are configured for monitoring and recording corresponding one or more respiration-related signals from the subject and generating measured data 104 indicative thereof. The system 100 includes a control unit 105, which is typically a computer system including inter alia a processor unit 106 and a data presentation unit (e.g. display) 108. The control unit 105 is connectable (via wireless or wired signal transmission) to the sensing unit 102 and to the therapeutic treatment tool (ventilator) 120. It should be understood that the sensing unit 102 may or may not be a constructional part of the monitoring system. The monitoring system 100 may be a computer system configured (preprogrammed) for identifying input coming from a specific type of sensing unit. The processor 106 is adapted (preprogrammed) to receive and process the measured data 104 to generate a corresponding time-varying energy function 110. The energy function may then be displayed on display 108 numerically, in the form of a graph, or in the form of a still or dynamic digital image. The measured data 104 is processed and displayed in form of graph and/or dynamic image. In some embodiments, the measured data 104 is collected by the sensing unit 102 during a 20 second recording and a grayscale video depicting the relative geographical distribution of respiratory sound is created. A sequential dynamic display of images is displayed 60 seconds after the start of the recording, generating a movie that shows changes occurring in the distribution of vibration energy across lung regions over time.

In some embodiments, a normalized dynamic image is displayed after each recording, and the raw data is stored digitally on the processor unit 106 for later review and analysis.

In accordance with this specific example of the invention the system 100 is used to manage the operation of a mechanical ventilator system 120 for ventilating a subject. Typically, ventilator system 120 feeds ventilated airflow parameters, including one or more of flow, volume and pressure waveform 122 to the processor and such ventilator waveform may then be fed to and displayed on display 108. For example, the sum of the vibration energy function in the lungs is calculated by the processor unit 106 during each breath cycle (inspiration and expiration) and is matched with each tidal volume (V_{T}), which is a parameter of the VC mode.

The acoustic-based sensing unit 102 may include a plurality of transducers producing each an analog voltage signal indicative of pressure waves arriving at the transducer (e.g. as described in WO 03/57037). The acoustic signals are transmitted to the processor unit 106. The analog signals are digitized by a multichannel analog to digital converter. For example, the processor unit 106 includes a 16-bit acquisition level and a sampling rate of 19.2 kHz that acquires the analog signals and converts them to digital data. The digital data signals P(xᵢ,t) thus represent the pressure wave at the location xi of the ith transducer (i=1 to N) at time t. The signals may be denoised by the processor unit 106 by filtering components having frequencies outside of the range of respiratory sounds, for example, vibrations due to movement of the individual, or cardiac sounds. Each signal may also be subject to band pass filtering by the processor 106 so that only components in the signal within a range of interest are analyzed. Therefore, the signals may undergo several stages of filtering that capture the frequency range of breath sounds (150-250 Hz) and therefore reduces interference generated by chest-wall movement and heart sounds.

The N signals P(xᵢ,t) from N acoustic transducers, where index i corresponds to a spatial coordinate or a sensor index, are divided into subintervals of length Δt. An input device may be used to input the time interval Δt. Alternatively, the time interval Δt may be determined automatically by the processor unit 106. The processor unit 106 calculates an average acoustic energy \tilde{P}(xᵢ,tⱼ, tⱼ+Δt) over each subinterval from tⱼ to (tⱼ+Δt), where, (tⱼ=(j−1)Δt), at the N locations xi in a calculation involving at least one of the signals P(xᵢ,t). The average acoustic energy \tilde{P}(xᵢ,tⱼ,tⱼ+Δt) is preferably determined as disclosed in U.S. Pat. No. 6,887,208. The functions \tilde{P}(xᵢ,tⱼ,tⱼ+Δt) are then summed with respect to x,

$${\sum }_{Xi} \tilde{P} ( {x}_{i} , {t}_{j} , {t}_{j} + Δ t )$$

in order to obtain a total average acoustic energy in the airways during the interval from tⱼ to Δtⱼ.

It should be noted that the so-called “weighted approach” can be used, the combined energy function Pₜₒₜₐₗ(j) can be:

$${P}_{total} ( j ) = {\sum }_{i} {W}_{i , j} \cdot P ( {x}_{i} , {t}_{j} , {t}_{j} + Δ t ) \qquad [ 1 ]$$

It should be noted that the weight W may be determined by independent means, e.g. coordinate dependent, or from time dependent analysis of each signal P_{i,} in both such cases the weight being only sensor dependent and not time dependent; or for more advances analysis, as presented in the above equation, the weight is both time and space dependent, for instance a statistical calculation that is designed to reflect the relevancy or quality of a signal obtained from sensor i during the time interval [tⱼ,tⱼ+Δt].

The signal obtained by each sensor is processed, including filtering, framing, etc., obtaining a set of signals, noted as P_{i,} that have been shown to be correlated with lung ventilation either by means of volume or flow. From this set, a sub set of sensors, containing as few as a single sensor and as many as the entire set, corresponding to any region of the lung is chosen, the weights W_{ij,} are calculated and the total power associated with respiration Pₜₒₜₐₗ(j) at this region is calculated following the above equation [1].

Thus, the processor 106 sums up or averages the individual energy functions from the plurality of sensors to obtain a combined energy function. The acoustic signals collected from the acoustic sensors are processed and the resulting average acoustic energy is calculated for each recording time period. The recorded time period is typically divided into sampling frames.

More specifically, the determination of the combined energy function consists of the following: The signal recorded by each acoustic sensor (microphone) corresponds to pressure waves that interact with the surface of the sensor. The source of these pressure waves is partly random ambient noise, partly thermal noise of the skin complying with a Boltzmann energy distribution, and partly sound associated with respiration. The latter can be separated into two general types: ventilation related sounds and additional lung sounds that are not directly ventilation related, namely wheezes and crackles. It should be understood that the energy function is a signal obtained from the raw measured signal after removing any other components by performing one of the following processing operations: time analysis, spectral analysis, adaptive morphological filtering, model aided analysis, etc.

The processed data is correlated with respiration, originated by transfer of momentum from the gas to the airways wall tissue via spontaneous collisions. These collisions are a mean of reducing gas kinetic energy essential to adapt flow profile to changes in tube radii and total cross section area of the bronchial tree. The rate of collisions depends on the following: gas flow rate, surface area for the interaction (inner radii of the airways), the momentary average kinetic energy of a unit volume of gas. Via such collisions the flowing gas is able to dissipate energy to the surrounding therefore reduce its mean velocity, while pressure gradient along the bronchial tree may still cause further acceleration or deceleration of the gas. As the tissue is at a higher energy state, the excessive energy dissipates further and resonates within the Rib-cage while transmitting sound to the environment which is then detected by the microphones. The energy function therefore reflects the momentary energy dissipated from the flow to lung tissue at a region of interest of the lung after decay and delay due to propagation through the thorax. Changes in any one of the above will be directly reflected by the energy function.

FIG. 2 shows such an energy function graph for a spontaneously breathing patient. This example relates to a 12 second recording from a 30 years old non-smoking healthy male volunteer. As can be seen, the graph typically has harmonically arranged patterns, higher amplitude for inspiration (I) and lower amplitude for expiration (E).

Specific characteristic values of the energy function such as: rise time, relaxation time, Inspiratory Vs' Expiratory energy, Inspiratory Vs' Expiratory length, number of ‘events’ per respiratory cycle, inter cycle similarity etc., can be calculated and used as an insight for flow and ventilation physiology of the specific recorded lung. These parameters can also be compared to typical values that correspond to respiration of a healthy lung at equivalent conditions. Such expected values can be obtained by either collecting clinical data from controlled studies or by means of dedicated model prediction, or a combination of both.

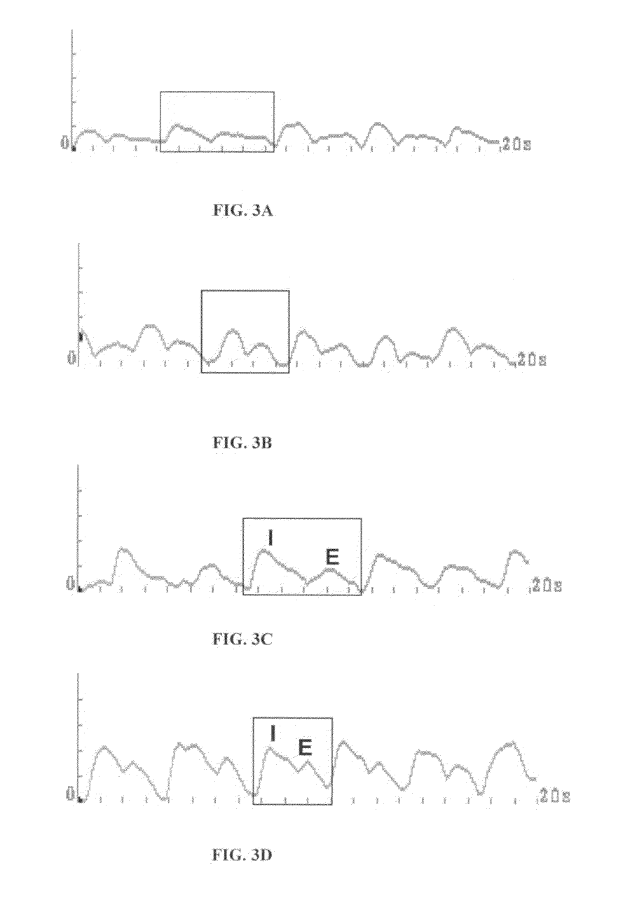

FIGS. 3A and 3B exemplify the energy function graph recordings before and after an inhalation treatment, respectively, from an asthmatic patient. During an asthmatic episode, airways at the middle generations of the bronchial tree tend to contract. The negative pressure gradient that drives respiration which is produced by expanding the pleura when contracting the trachea is not sufficient to overcome the increased airways resistance. As a result, flow is dramatically reduced during both inspiration and expiration. Therefore respiration becomes very shallow and when patient is requested to take deep breathes as during the energy function recording, respiratory rate is very low. In addition, the lung itself is continually inflated, resulting in a longer delay when the energy dissipates through the lung tissue and longer response times of the signal recorded at the surface. This is noted as smearing of each of the flow events (namely inspiration and expiration).

As this is a spontaneous breathing scenario, the dominant affect is reduced flow as described above. However, in a similar scenario but under mechanical ventilation, where the flow is controlled, airways restriction will result in higher mean velocity and a higher wall surface to volume ratio. These effects will appear as enhanced energy function signals, and fast rise time though inspiratory and expiratory peaks might still merge depending on the ventilator flow profile.

FIGS. 3C and 3D exemplify the energy function graph recordings before and after a physiotherapy treatment, respectively, from a spontaneously breathing individual.

The energy function graph is not the only tool that can be used when comparing ventilation efficiency (either spontaneous or under mechanical ventilation), and the mode of ventilation. The separation of inspiratory and expiratory peaks in spontaneously breathing and mechanically ventilated patients during different modes of mechanical ventilation should preferably also be considered.

Energy function graphs recorded on mechanically ventilated patients are different than graphs obtained on spontaneously breathing patients. The following parameters may influence the profile of the graph:

Ventilator settings (mode of ventilation, flow rate, rise time, I:E ratio, pressure limits, inspiratory time, and possibly PEEP)

Waveforms

Respiratory holds

Patient-ventilator interaction

There are several ventilation modes that are used for maintenance of mechanical ventilation in patients with similar clinical abnormalities. The most common are assist volume control (VC), assist pressure control (PC) and pressure support (PS) modes.

In VC, tidal volume (V_{T}), respiratory rate (RR) and inspiratory flow rate are set by the ventilator. Waveforms are either squared (VCsq) or decelerating (VCdec). In PC, pressure, RR and inspiratory time are set. In PS, the level of added pressure for inspiration is set and all other parameters are determined by the patient according to his or her condition.

The following are experimental results showing a series of energy functions (in a sampling frame rate of 0.17 second) obtained from several patients, each ventilated in different modes of mechanical ventilation. As described below, the energy function graph varies according to the ventilation mode and various features of the graph are characteristic of certain modes. In VCsq, flow is increased very sharply at the beginning of inspiration and stays constant during the rest of inspiration. Full expiration directly follows full inspiration. In VCdec, flow is increased very sharply at the beginning of inspiration and is slowly decelerated during the rest of inspiration. The flow in PC and PS is very similar to the flow in VCdec (sharp increase at the beginning followed by slow deceleration). In PS, inspiration ends when flow is 25% of maximal.

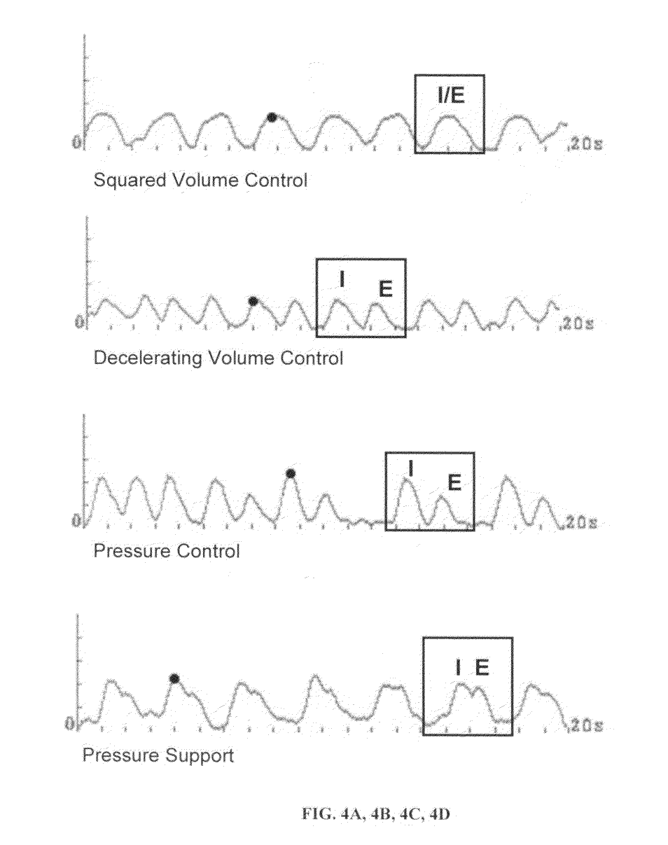

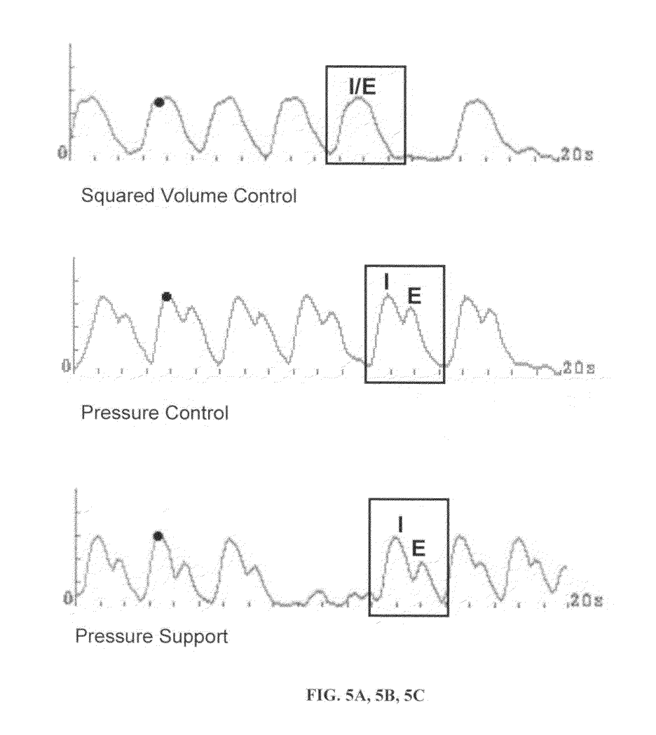

FIGS. 4A-4D show the measured energy functions graphs for the same patient under mechanical ventilation with, respectively, VCsq, VCdec, PC and PS modes. As shown in FIG. 4A (VCsq), in this specific example, inspiration and expiration are so close that they form a single peak. Moreover, energy is lower at the beginning of the peak (inspiration) than at the end (expiration). This is likely due to the relatively lower inspiration flow rate in VC. In FIG. 4B (VCdec), the inspiration and expiration peaks are similar and well separated. In FIG. 4C (PC), energy during inspiration is typically higher than during expiration, revealing a relatively higher initial inspiration flow rate in this mode. As can be seen in FIG. 4D (PS), inspiration and expiration peaks are closer than in PC because of the residual flow at the end of inspiration in this mode.

FIGS. 5A-5C exemplify the energy function graphs of a patient mechanically ventilated in VCsq, PC and PS modes, respectively.

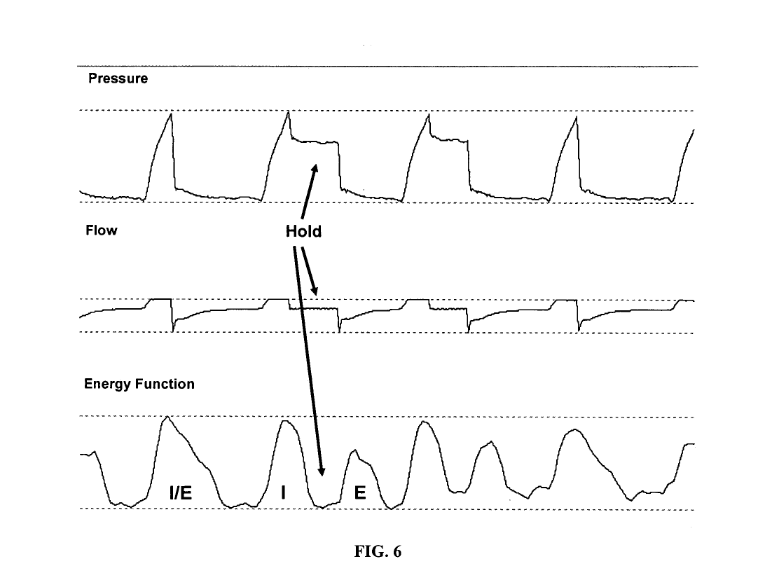

FIG. 6 shows the pressure, air flow and energy function graphs for another patient under the VCsq ventilation mode. In this example, during one of the respiratory cycles, a hold was performed between inspiration and expiration. As a result, the inspiration and expiration peaks, which are typically combined in a normal VCsq (see above), were separated. Respiration hold allows confirming accurate synchronization of the ventilator waveform and the energy function graph. Holds validate the use of the energy function graph as source of information on breathing.

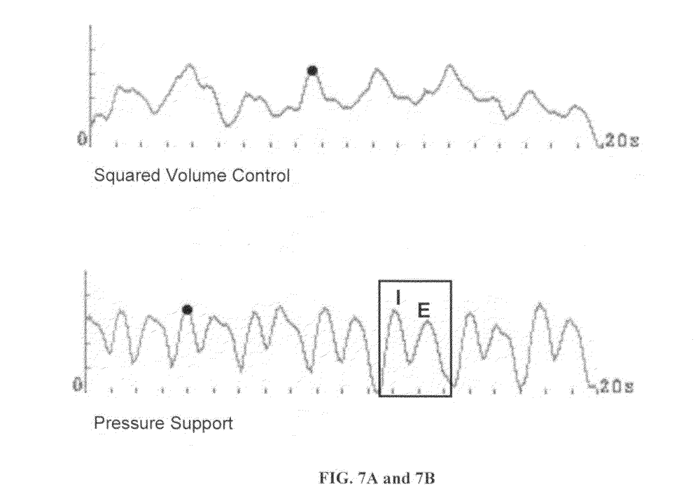

Generally, patient-ventilator interaction (PVI) occurs when the ventilator cycles are out of phase with the patient's respiratory muscle activity. Dyssynchrony causes discomfort and unnecessary inspiratory and expiratory work. PVI may generate a disharmonious energy function graph where respiratory cycles are not easily identifiable. FIGS. 7A and 7B display two energy function graphs recordings obtained from the same patient while under VCsq and PS ventilation modes, respectively. For this particular patient, the vibrations recorded on VCsq are less harmonious than those recorded on PS. This last mode seems therefore more beneficial in this case.

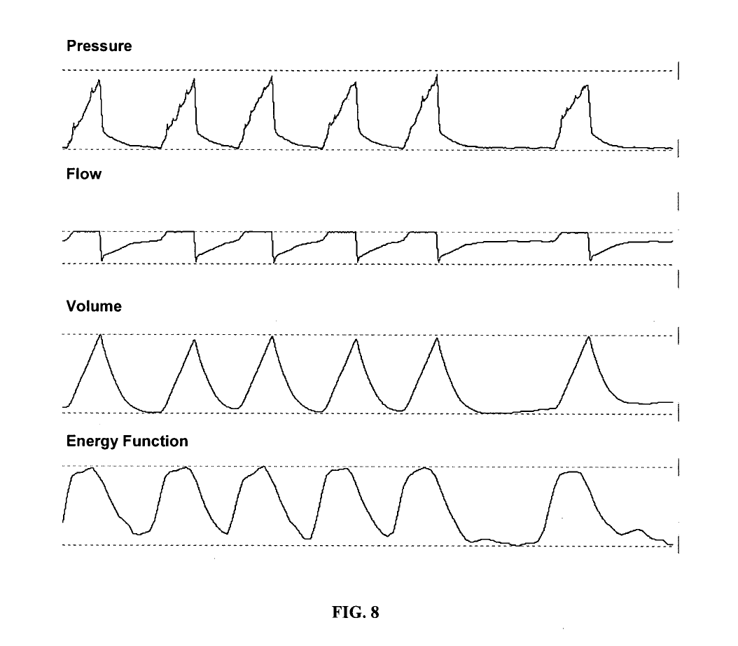

In order to better understand the energy function recorded on mechanically ventilated patients, synchronization with the ventilator waveforms (pressure, flow and/or volume) is important. Data can be directly collected from the ventilator, or can be universally sampled while inserting a commercially available flow sensor in the disposable breathing system of the patient. The different waveforms for the pressure, air flow, volume and the energy function graph can be synchronized and displayed as shown in FIG. 8.

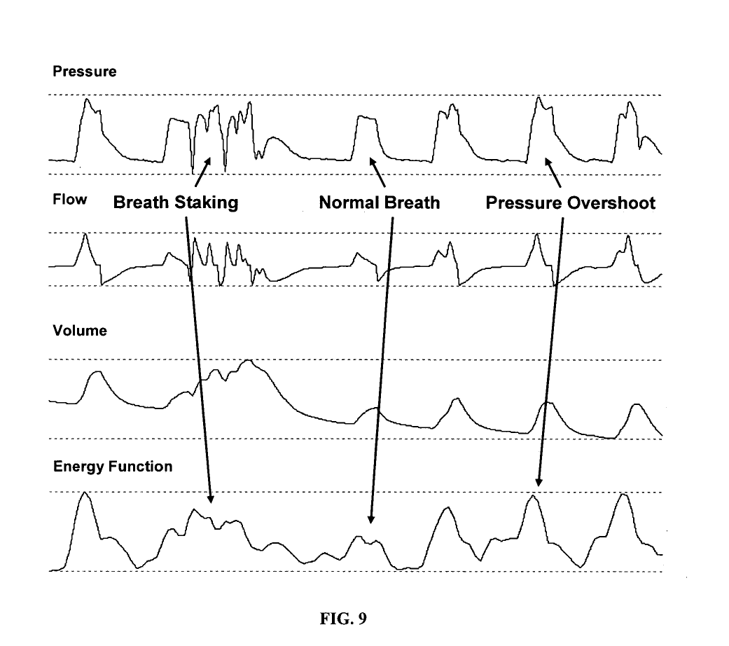

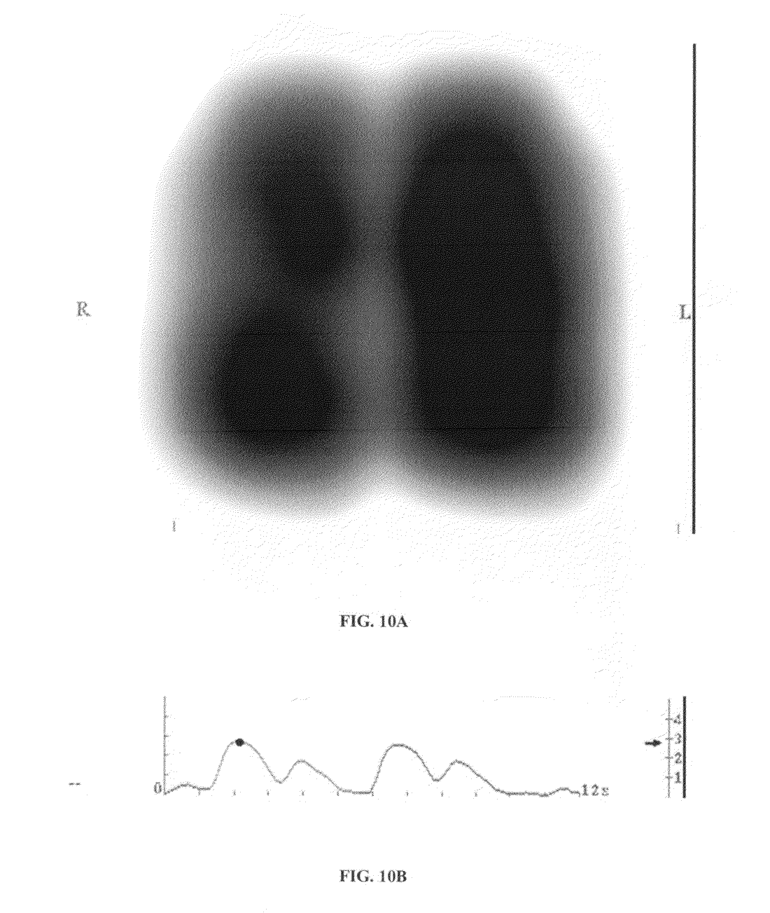

Synchronization allows to better understand the energy function graph and to detect changes in acoustic energy related to changes in flow or pressure such as exemplified in FIG. 9. This figure is showing a case of breath stacking (three breaths one right after the other without allowing time for expiration, thus causing excess volume). The one normal breath in this figure is the one in the middle of the recording where the pressure waveform is a plateau during inspiration. Pressure overshoot can be seen in the first and the last three breaths where there is a spike in the pressure instead of the plateau. The results in the spikes are seen on the flow and energy waveforms.

Reference is made to FIGS. 10a and 10b illustrating experimental results, where FIG. 10a shows a normalized digital image representing a mid-inspiration frame of a representative respiration cycle of a 12 seconds recording obtained from a 30 years old non-smoking healthy male volunteer. This is a vibration response image representing the energy function measured from different regions of the lungs. FIG. 10b illustrates an energy function graph produced from the same measured data of FIG. 10a indicative of the average vibration energy as a function of time throughout the respiratory cycle.

In some embodiments, the energy function graph enables an appropriate selection of the dynamic image frame recordings allowing an accurate diagnosis and selection of appropriate ventilation mode and parameters.

In some embodiments, the image used for analysis is a maximal energy frame (MEF), which provides the most information on the distribution of lung vibration, is selected in the range of frames. The MEF usually approximates peak inspiration. A larger image indicates a more homogeneous distribution of vibration intensity throughout the lung and a smaller image a more focal distribution. The total output from all the sensors is presented as an intensity bar and graph over time. Each subject's recording has different high and low value areas within each respiratory cycle, according to the vibration intensity.

In some embodiments, MEF areas and vibration energy are compared enabling straightforward quantification. MEFs are extracted from normal, regular, and consistent cycles available within each 20-second recording. Artifact-free MEFs are extracted a priori from these selected cycles according to predefined rules and criteria listed below. The MEF area of the dynamic image is measured. Regional areas are obtained by first separating the image into three regions on the basis of the rows of sensors (upper: rows 1 and 2; middle: rows 3 and 4; and lower: rows 5 and 6). Each segment is then measured. Because the position of the sensors is kept the same for each image recorded on a given patient, the three regions are standardized across studies.

The regional vibration energy, which is not affected by normalization of the image, is also analyzed. Vibration intensity is computed in units of energy (watts×constant), reflecting the acoustic energy associated with respiration. The vibration energy is derived from the signal at each of the sensors as follows: the digitized acoustic signals are bandpass-filtered between 150 and 250 Hz to remove heart and muscle sounds; median filtering is applied to suppress impulse noise, and truncation of samples above an automatically determined signal-to-noise threshold is performed. The resulting signal is down-sampled to produce the vibration energy.

In some embodiments, the energy function graph enables an appropriate selection of the dynamic image frame recordings allowing an accurate diagnosis and selection of appropriate ventilation mode and parameters.

It should be noted that the frames can be selected from the synchronization between the ventilator waveform and the energy function graph by using flow/pressure ventilator information. Otherwise, the frames may be selected a priori from the recordings on the basis of the predefined rules and criteria exemplified below:

1. To correctly characterize respiratory cycles, the following criteria may be applied:

Vibration intensity is lower between two cycles (from expiration to inspiration) than within a same cycle (from inspiration to expiration).

The distance between expiration and the next inspiration in the energy graph is greater than the distance between inspiration and expiration within the same cycle.

The area of rapidly increasing vibration from baseline indicates inspiration.

2. To correctly identify inspiration within a respiratory cycle, these criteria may be applied:

The first dramatic rise of vibration in a cycle is inspiration.

If there is no separation between inspiration and expiration in the energy graph, inspiration is considered to end at the peak signal.

If there is more than one peak in the cycle, the first peak is considered the maximal inspiration signal.

If there is a hint of separation in the form of a shoulder in the energy graph, the shoulder is considered an inspiration.

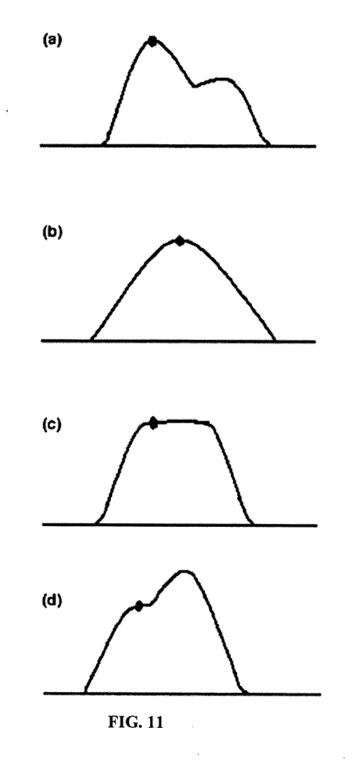

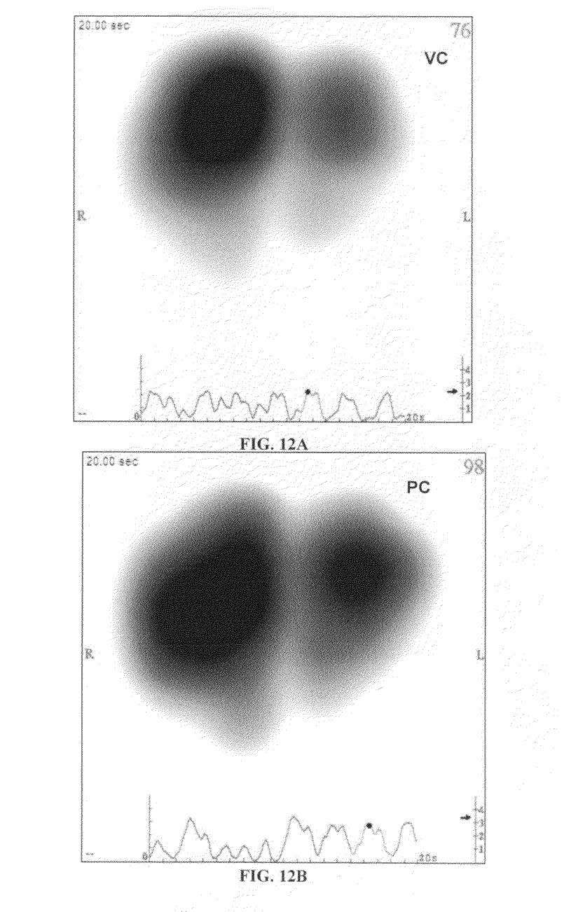

3. The following criteria may be applied in choosing the maximal inspiration frame (FIGS. 11a-11d);

The frame with the maximal energy within inspiration is chosen for analysis.

If inspiration and expiration are clearly separated, the MEF during inspiration (first peak) is chosen (FIG. 11a).

If inspiration and expiration merge into one peak in the waveform, the frame closest to that peak is chosen from the image (FIG. 11b).

If inspiration and expiration form a plateau, the first frame at zero slope is chosen (FIG. 11c).

If there is no peak and the shoulder is curvilinear, the frame nearest the inflection point is chosen (FIG. 11d).

4. The following criteria may be applied in choosing the range for normalization of recording:

The dynamic image is produced by the control unit and is normalized based on a chosen range of frames. The MEF at inspiration is selected for analysis.

The chosen frame is that having the highest energy in the range chosen.

If there is a peak in the waveform, the chosen range consists of the two frames before and two frames after the peak. If this captures a frame with energy greater than the chosen frame, only frames with energy less than the chosen frame are included.

If there is no peak and only a shoulder, the chosen range consists of the two frames before and the chosen frame. In some embodiments, there is provided a novel method for controlling mechanical ventilation using the combination of an acoustic image and an energy function as a feedback signal. The controlling is aimed at selecting the optimized mode or a set of optimized parameters in a specific mode. The selection of the set of parameters within a mode can generally be performed by using either one or both of the acoustic image and the energy function as the feedback signal.

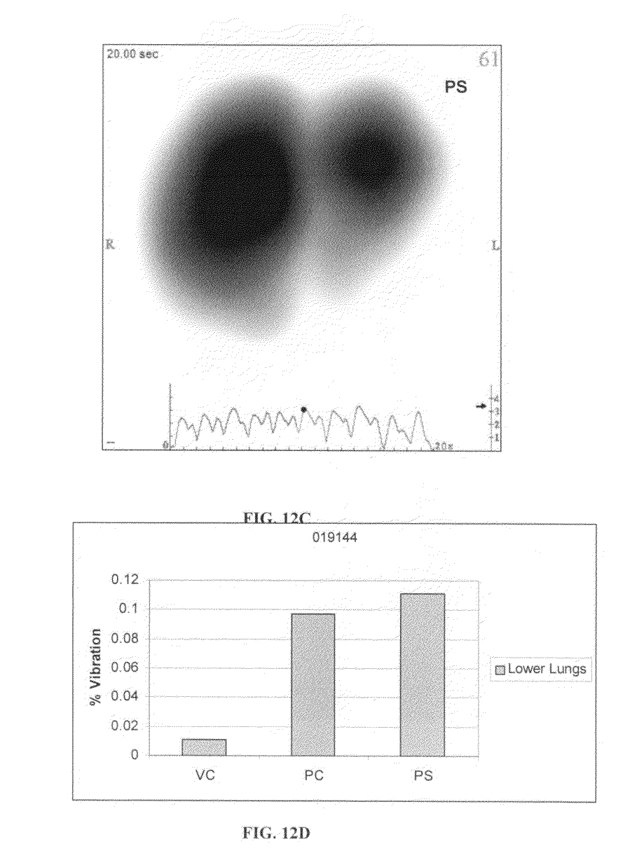

Reference is made to FIGS. 12A-12C, representing successive vibration response images recordings of a mechanically ventilated patient during different modes of mechanical ventilation. These three images were recorded on a patient ventilated in three different modes of mechanical ventilation, respectively, while tidal volume was held constant: Volume Control (VC), Pressure Control (PC) and Pressure Support (PS). In addition, the quantification graph, illustrated in FIG. 12D, reveals that the vibration energy in the lower regions is increased in PC and PS modes as compared to VC mode. The maximal energy frames were extracted from recordings of a 73 year-old mechanically ventilated female with respiratory failure secondary to pancreatitis.

The correlation of vibration energy and airflow in the lungs supports the premise that the increase in vibration distribution in a particular lung area (example: the lower lung regions) during measurement with one set of mechanical ventilation parameters (mode, PEEP, RR, pressure, etc) when compared to a measurement recorded within a short time period and obtained with another set of mechanical ventilation parameters, is correlated strongly with an increase in flow in these regions. Because V_{T} values were held constant in this particular example, these results suggest that the distribution of airflow in the lower lung regions is greater in PC and PS compared to VC. The regional area analysis demonstrates that the increase in the total area is due to the expansion of the lower lung region whereas areas in the upper and the middle regions decreased.

When comparing VC to PC and to PS, the data showed a shift in image area away from the upper lung regions toward the lower.

The regional vibration intensity values calculated from signals recorded in the three modes showed similar trends. There is a significant percentage increase in vibration intensity values in the lower regions. The relative increase in vibrations in the lower region in PS versus VC is statistically significant (p<0.05). Here again, a shift of vibration toward the lower lung regions is noted.

Therefore, the method of the present invention enables to determine a correlation between the parameters of the different modes (e.g. V_{T} values) and the vibration energy in patient. Holding RR constant as V_{T} increases, the total lung vibration measured with the technique of the present invention increases with airflow.

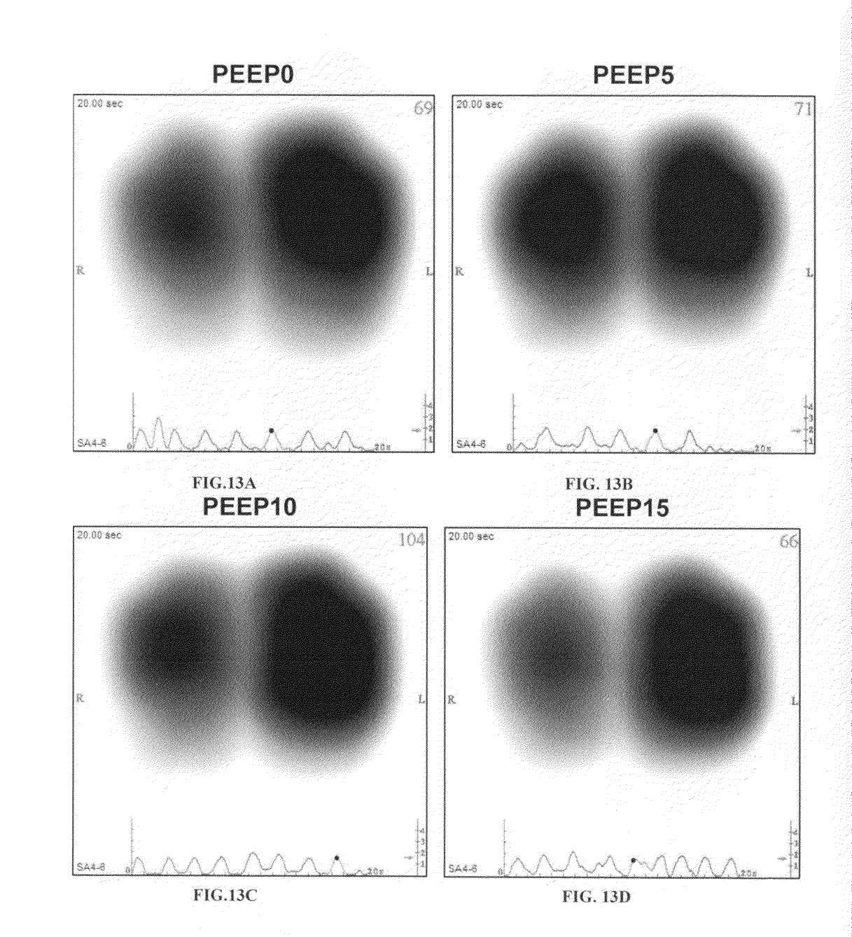

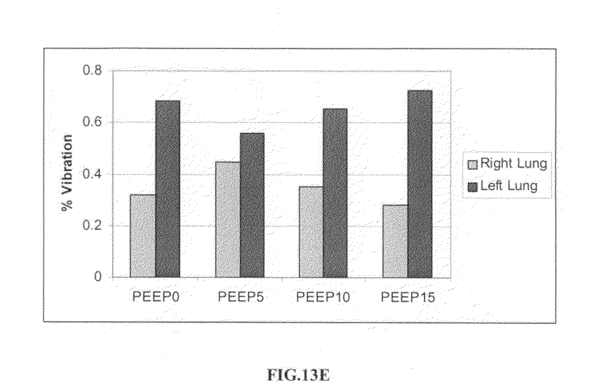

FIG. 13A-13D show the experimental results indicative of the effect of PEEP changes on vibration imaging response obtained from a 77 year old male suffering of myasthenia gravis. Each of these figures shows the vibration imaging response and the corresponding energy function graph. The vibration imaging response recordings are performed on this patient at four levels of PEEP: 0, 5, 10 and 15 cm H₂O. As revealed in these images, the vibration energy in the right lung is maximal at PEEP 5 (FIG. 13B) and decreased at lower and higher PEEP levels. It should be noted that the decrease of lung vibration is indicative of the air saturation condition. In addition, the quantification graph illustrated in FIG. 13E reveals more energy balance between the lungs at PEEP 5 (left lung: 56%, right lung: 44%) when compared to other PEEP levels. Thus, the present invention provides a novel, effective technique for monitoring the respiratory system of a subject to enable controlling procedures (e.g. therapeutic treatment) of the kind affecting the operation of the subject's respiratory system.

The technique of imaging of the present invention may also be used to assess asymmetric lung disease in patients. The following are experimental results obtained from consecutive intensive care unit (ICU) patients with one diseased lung on chest radiograph, and from ICU patients with normal chest radiograph. It should be noted that in the ICU, the most conventional methods for assessing the lungs are chest radiography (assessment of anatomy) and auscultation (assessment of lung sounds). Chest radiography is associated with some radiation exposure and is not practical for frequent assessment of lung pathophysiology in an ICU setting. Auscultation is simple and useful but suffers from its subjective nature. In patients with asymmetric lung disease, the diseased lung usually appeared as irregular, smaller and lighter in color (reduced vibration signal) that the non-affected lung. In patients with normal chest radiographs, the right and left lungs developed similarly dynamic image of distribution of vibration responses, and the percent lung vibrations from both side were comparable (53±12% and 47±12%, respectively). In ICU patients with asymmetric lung disease however, the percent lung vibrations from the diseased and non-diseased lungs were 27±23% and 73±23%, respectively (p<0.001). It should be noted that vibrations from breathing are the dominant signals and typical background ICU noise generally has no or minimal effect on the recording.

Analysis of the image can be performed by comparing the weighted pixel count analysis from both lungs. Digital analyses of images reveals that the percentage of weighted pixel counts and the percentage of the total vibration are reduced in patient having affected lungs. In this method, the pixels making up the image were assigned values based on their color with the darker pixels assigned higher values. The weighted pixel count from diseased and non-diseased lungs were 33±21% and 67±21%, respectively (p<0.003). Therefore, the technique of the present invention, provide a radiation-free method in identifying and tracking of asymmetric lung parenchymal process in patients during their ICU stay. The technique of the present invention is non-invasive and, unlike auscultation, is objective and does not depend on the auditory acuity of the clinician as it provides a visual display of distribution of lung vibrations.

Those skilled in the art will readily appreciate that various modifications and changes may be applied to the embodiments of the invention as hereinbefore described without departing from its scope defined in and by the appended claims.

1. A method for use in monitoring the respiratory system of a subject, the method comprising:
(a) recording one or more signals from the subject, the signal varying in time according to operation of the respiratory system; and;
(b) processing the recorded signals to obtain a predetermined functional thereof presenting one or more time-varying energy functions of the subject, an abnormality in said one or more energy functions being indicative of a suspected abnormality in the operation of the respiratory system.

2. A method according to claim 1, wherein the signals are acoustic signals.

3. A method according to claim 2, wherein the signals are recorded by a plurality of acoustic sensors placed over the subject's thorax or back, and said at least one time-varying energy function is obtained from one or more specific regions of lung.

4. A method according to claim 3, comprising summing or averaging the time-dependent acoustic signals of the plurality of sensors indicative of the whole lungs.

5. A method according to claim 1, wherein the one or more energy functions of the subject is/are displayed in form of one or more graphs.

6. A method according to claim 1, wherein the one or more energy functions of the subject is/are displayed in form of still or dynamic digital image, or in form of succession of still images or frames.

7. A method according to claim 6, wherein the still or dynamic digital image is indicative of the regional distribution of vibrations in the lungs.

8. A method according to claim 6, comprising analyzing said dynamic image thereby providing data indicative of the intensity and distribution of vibration within lungs in real-time.

9. A method according to claim 1, wherein the one or more energy functions of the subject is/are displayed in form of graph and in form of image.

10. A method according to claim 9, comprising analyzing said at least one energy function graph to select an appropriate still image in a dynamic image recording.

11. A method according to claim 1, wherein said processing of the one or more energy functions comprises determining a degree of correlation between one or more parameters of the energy function and one or more corresponding parameters of certain reference energy function.

12. A method according to claim 11, wherein said one or more parameters of the energy function include at least one of the following: geographical distribution and/or intensity of energy, synchronization and/or balance between lungs, signal periodicity and/or signal symmetry of the function.

13. A method according to claim 1, comprising utilizing results of said processing of the energy function for optimizing the operational mode of mechanical ventilation.

14. A method according to claim 1, comprising utilizing results of said processing of the energy function for selecting optimized parameters set for a specific mode.

15. A method according to claim 13, comprising utilizing results of said processing of the energy function for selecting optimized parameters set for a specific mode.

16. A method according to claim 14, wherein said one or more optimized parameters set for a specific mode include at least one of the following: respiratory rate, inspiratory pressure, pressure support level, tidal volume, levels of PEEP.

17. A method according to claim 15, wherein said one or more optimized parameters set for a specific mode include at least one of the following: respiratory rate, inspiratory pressure, pressure support level, tidal volume, levels of PEEP.

18. A method according to claim 1, wherein said processing of the energy function comprises image analysis.

19. A method according to claim 18, wherein said image analysis comprises characterizing a least one of different modes of ventilation and different parameter sets for a specific mode of ventilation, by different geographical distribution of vibration in the lung.

20. A method according to claim 1, wherein said processing of the energy function comprises extracting a maximal energy frame (MEF) indicative of a frame providing the most information on the distribution of lung vibrations in a selected range of frames.

21. A method according to claim 1, comprising use of results of said processing to control operation of a respiratory ventilator.

22. A method according to claim 21, wherein said control comprises changing between different ventilator settings including at least one of the following: ventilation modes, respiratory rate, inspiratory pressure, pressure support level, tidal volume, flow rates, rise times, I:E ratios, pressure limits, inspiratory times, and levels of PEEP.

23. A method according to claim 22, comprising synchronizing a ventilator waveform and the energy function.

24. A method according to claim 1, comprising using results of said processing of the energy function for quantifying the lung vibration in a particular region of interest by using a quantification method comprising determining the percentage contribution of lung regions.

25. A method according to claim 24, wherein said quantification method comprises quantifying the lung vibration by determining the weighted pixel count of image.

26. A method according to claim 1, comprising using results of said processing of the energy function for assessing lung disease in patients.

27. A system for monitoring the respiratory system of a subject, the monitoring system comprising:
a control unit configured for receiving data indicative of one or more respiration-related signals, and configured and operable for processing the received data and generating at least one corresponding time-varying energy function and displaying said at least one energy function, and being configured and operable for using said at least one corresponding time varying energy function for determining at least one of the following: a condition of the respiratory system, an optimal operational mode or optimal parameters set for a specific operational mode of a ventilation system being applied to a subject.

28. A system according to claim 27, comprising a sensing unit comprising one or more sensors for recording corresponding one or more respiration-related signals from the subject, generating data indicative thereof to be processed by the control unit.
